(19) United States
(12) Patent Application Publication
(10) Pub. No.: US 2023/0337918 A1
(43) Pub. Date: Oct. 26, 2023

(54) Novel Treatment and Imaging Methods for Early Detection of Diseases

(71) Applicant: Ishtar Faisal Algburi, London (CA)

(72) Inventor: Ishtar Faisal Algburi, London (CA)

(21) Appl. No.: 17/098,380

(22) Filed: Nov. 14, 2020

(51) Int. Cl. A61B 5/00; A61B 5/055; A61K 31/07; A61K 31/375; A61K 31/355; A61K 31/05; A61K 31/343; A61K 31/352; A61K 31/12; A61K 31/155; A61K 31/138; A61K 31/5377

(52) U.S. Cl. CPC A61B 5/0036; A61B 5/055; A61B 5/0042; A61B 5/4842; A61K 31/07; A61K 31/375; A61K 31/355; A61K 31/05; A61K 31/343; A61K 31/352; A61K 31/12; A61K 31/155; A61K 31/138; A61K 31/5377

(57) ABSTRACT

This invention relates to treatment and detection of the diseases by Magnetic Resonance Imaging. Previously, there was no way to use MRI imaging to early detect and map the agent bind to hypoxia in tissue, blood brain barrier leakage or break down, tissue under glycolysis, brain activation in neurodegenerative diseases, aggressive cancer, senescent tissues, and T-cells senescent. Also, novel methods to treat neurodegenerative diseases, aging, senescent tissues and T-cells senescent. Embodiments of the present invention use novel treatment and imaging methods are disclosed for early detection and mapping of diseases by using MRI. The endogenous of the biological tissue and exogenous agents can be used to produce MRI contrast which rely on the endogenous proteins and exogenous protons, as a source of the contrast, such as hydroxyl, amine, and amide protons, and thereby provide imaging and mapping for the early detection of hypoxia in tissue, blood brain barrier leakage or break down, tissue under glycolysis, brain activation in neurodegenerative diseases, aggressive cancer, senescent tissues, T-cells senescent, and other diseases by using endogenous and exogenous protons contrast by using MRI. Also, this invention relates to novel methods to treatment neurodegenerative disease, senescent cells and T-cells senescent.

### CROSS-REFERENCE TO RELATED APPLICATION

This application claims the benefit of priority to U.S. Provisional Patent Application No. 62/937,257, filed on Nov. 19, 2019, the entirety of which is incorporated by reference for any and all purposes.

### BACKGROUND

Embodiments of the invention described in this specification relate generally to disease detection by imaging, and more particularly, to novel treatment and imaging methods for early detection of diseases using chemical exchange saturation transfer (“CEST”) magnetic resonance imaging (“MRI”).

There is no current method used for early detection of neurodegenerative diseases by using MRI before 10-20 years of the symptoms appear. The method presented in this invention can detect neurodegenerative diseases by using CEST MRI by using endogenous proteins and exogenous protons contrast which can be enhanced by glucose with or without monocarboxylic transporters (MCTs) combined with or without sodium-proton exchanger (Na⁺/H⁺).

Positron emission tomography (PET) scanners are used for mapping hypoxia in the tissue of living people. Hypoxia is a condition of insufficient oxygen to support metabolism which occurs when the vascular supply is interrupted. PET scanners map hypoxia also by tracers or contrast agents that are injected into the bloodstream. PET tracers have been used for the identification of hypoxia in living tissues and solid tumors by marking the hypoxia with emissions of mild radioactivity. Tumor hypoxia is the result of an inadequate supply of oxygen to tumor cells. Detection of hypoxia in tumors is of greater clinical relevance because tumor aggressiveness, metastatic spread, failure to achieve tumor treatment, and increased rate of recurrence are all associated with hypoxia. Tumor hypoxia increases resistance to radiotherapy and chemotherapy, resulting overall in poor clinical prognosis. Thus, in vivo measurement of tumor hypoxia could be helpful to identify patients with worse prognosis or patients who could benefit from appropriate treatments, such as radiation therapy or chemotherapy.

Blood brain barrier (BBB) leakage or breakdown early in many diseases such as cancer, AD, PD, HD, ALS, MS, HIV-1-associated dementia and other diseases. Also, BBB is identified as potential early warning sign for neurodegenerative diseases and associated with inflammatory and immune responses and there are potential connections between blood brain barrier dysfunction and several neurodegenerative diseases, including Alzheimer's and Parkinson's diseases, HD, ALS, MS and other disease. Increase permeable blood brain barrier may allow more toxic amyloid and tau proteins to enter the brain. BBB breakdown can be used to early detection of the diseases. In most cases, current method now to detect BBB leakage or breakdown by using MRI contrast agent, such as Gadolinium, to better identify the blood brain barrier leakage. However, Gadolinium is expensive and has many side effects on humans, especially to humans with kidney diseases.

Multiple sclerosis (MS) is a disabling disease of the brain and central nervous system. Magnetic resonance imaging (MRI) can diagnose MS and monitor disease progression by using T₁-weighted brain MRI, but at a late stage, MRI cannot detect MS at an early stage. Some people with clinically-definite MS do not initially show lesions on MRI at the time of diagnosis. Also, MRI uses a contrast agent or tracer to detect active inflammation; this scan will highlight the new lesions or lesions that are growing. Also, these MRI methods are unable to detect small lesions; MRI scans can detect damage in the central nervous system.

In most cases, it helps to use a contrast agent, such as gadolinium, to better identify the lesions. However, gadolinium is expensive and has many side effects on humans, especially to humans with kidney diseases, also, cannot detect the lesions early. There is also evidence showing that activation of glial cells, including microglia and astrocytes, plays an important role in the inflammatory signalling in the diseases such as neurodegeneration diseases. Also, activation of microglia by amyloid beta, tau protein, and APP also results in an upregulation of inflammation. Method of imaging brain activation and used this method for early detection of the diseases in AD, PD, HD, ALS, MS, cancer and all other neurodegenerative disease by administration of glucose with or without MCTs inhibitors combined with or without Na⁺/H⁺ inhibitors.

Neuroinflammation results in a significant decrease intracellular pH. Nervous system diseases, such as, traumatic brain injury, Parkinson's disease (PD), epilepsy, ischemic stroke, and Alzheimer's disease (AD), Multiple sclerosis (MS), Huntington's disease (HD), the common characteristics are decreased pH or acidosis, aging or ischemia may cause intracellular and extracellular acidification. This acidification not only induces apoptosis but also alters enzyme activities and promotes the diseases. Methods of treatment nervous system diseases by increase of intracellular pH and extracellular pH are presented in this invention.

Cellular senescence and/or T-cells senescent are an aging mechanism that has been included in many age-related diseases and is cause of tissue dysfunction. senescence-associated secretory phenotype (SASP)-mediated tissue damage. Therapeutic targeting of an aging mechanism such as cellular senescence may have an impact on disease and more effective in preventing the progression of aging diseases. Currently there is no method to imaging cellular senescence and senescent tissue in vivo by using MRI, also, there is currently no effective way to remove or kill the senescent cells and T-cells senescent. This invention can image and detect senescent tissue by MRI and monitor the senescent tissue respond to the treatment by using non invasive MRI method.

Therefore, what is needed is a way to use MRI imaging to detect and map hypoxia in tissue, blood brain barrier leakage or break down, multiple sclerosis (MS), concussion, and traumatic brain injury (TBI), tissue under glycolysis, brain activation in neurodegenerative diseases, senescent tissues, T-cells senescent, and other diseases by using endogenous and exogenous protons contrast via MRI as opposed to PET scans which are longer in time, not as safe as CEST MRI, and provide lower resolution images than CEST MRI, and to do so without the need to inject an expensive and unsafe contrast agents or tracers.

### BRIEF DESCRIPTION

Novel treatment and imaging methods for early detection of disease are disclosed. In some embodiments, the Novel treatment and imaging methods for early detection of disease includes a plurality of steps comprising (i) acquiring, by way of a magnetic resonance imaging (MRI) machine, a T₂-image as an anatomical image, (ii) acquiring, by way of the MRI machine, a CEST image at S_{outside range} as a reference image, (iii) acquiring, by way of the MRI machine, a plurality of CEST images at S_{within range}, (iv) normalizing signal intensities of the acquired CEST images (S_{outside range} and S_{within range}) to al the reference image, (v) calculating a contrast difference in S (ΔS contrast range=S_{outside range}−S_{within range} for each image in the plurality of CEST images at S_{within range}), (vi) (ΔS contrast ratio=S_{outside range}/S_{within range} for each image in the plurality of CEST images at S within range, and (vii) detecting disease and mapping disease severity based on the calculated ΔS contrast. In some embodiments, the non-invasive CEST MRI imaging method for early detection of disease and mapping of disease measures a chemical shift of the reference image>15 ppm or <−15 ppm. For example, the non-invasive CEST MRI imaging method for early detection of disease and mapping of disease can use 16 ppm or 20 ppm downfield or use −16 ppm or −20 ppm upfield.

In some embodiments, the non-invasive CEST MRI method for early detection of neurodegenerative diseases and mapping the severity of neurodegenerative diseases includes a plurality of steps comprising (i) acquiring, by way of an MRI machine, a T₂-image as an anatomical image of the brain, (ii) acquiring, by way of the MRI machine, a CEST reference image at S_{outside range}=16 ppm, (iii) acquiring, by way of the MRI machine, a plurality of CEST images at S_{within range}=3.5 ppm or 3.4 ppm, (iv) normalizing signal intensities of the acquired CEST images (S_{outside} and S_{within range}) to the reference image at S_{outside range}=16 ppm, (v) calculating a contrast difference in S (ΔS contrast range=S_{outside range}−S_{within range} for each image in the plurality of CEST images at S_{within range}), and/or (vi) calculating a contrast ratio in S (ΔS contrast ratio=S_{outside range}/S_{within range} for each image in the plurality of CEST images at S_{within range}), and (vii) detecting early neurodegenerative diseases and mapping the disease in the tissue of the brain based on the calculated ΔS contrast and/or ΔS contrast ratio.

Novel treatment and imaging methods for early detection of disease of the present disclosure are based on the use of CEST MRI, not PET and without radiotracers. The endogenous magnetic resonance image (MRI) contrast of the biological tissue can rely on the endogenous protons of the proteins and peptides, also, exogenous protons of the agents as a source of the contrast, such as hydroxyl, amine, and amide protons, and thereby provide imaging and mapping for the early detection of the hypoxia in tissue, blood brain barrier leakage or break down, tissue under glycolysis, brain activation in neurodegenerative diseases, aggressive cancer, senescent tissues, T-cells senescent, and other diseases and predict response to the treatment.

The novel treatment and imaging methods for early detection of diseases using CEST MRI of the present disclosure may produce or result in the following elements. This list of possible constituent elements is intended to be exemplary only, and it is not intended that this list be used to limit the novel treatment and imaging methods for early detection of diseases and disease mapping using CEST MRI of the present application to just these elements. Persons having ordinary skill in the art relevant to the present disclosure may understand there to be equivalent elements that may be substituted within the present disclosure without changing the essential function or operation of the novel treatment and imaging methods for early detection of diseases and disease mapping using CEST MRI.
1. CEST image (the reference image at S_{outside range}, or S_{outside agent post})
2. CEST images (multiple images at S_{within range}, or S_{within agent post})
3. Difference in magnetization (ΔS _{contrast range}=S _{outside range}−S_{within range}), and/or ratio of magnetization (ΔS _{contrast ratio}=S_{outside range}/S_{within range})
4. Difference in magnetization after administration of glucose with or without MCTs inhibitors combined with or without administration of Na⁺/H⁺ exchange inhibitors (ΔS _{post contrast images}=S_{outside post}−S_{within post}) and/or ratio of the magnetization (ΔS _{post ratio}=S_{outside post}/S_{within post})
5. Difference in magnetization after administration of agents (ΔS_{agents} contrast images=S_{outside agent post}−S_{within agent post}) and/or ratio in magnetization (ΔS_{agents ratio} contrast images=S_{outside agent post}/S_{within agent post})
6. (MTR) asymmetry-agents post, acquiring CEST images at specific chemical shifts and at frequencies that decrease the magnetization of the exchangeable protons (Δω) of the administrated agents and also acquiring CEST images at specific chemical shifts at the opposite side of the same frequency (−Δω) which is decrease the magnetization of the exchangeable protons of the administrated agents at frequency (−Δω) according to MTR_{asym-agent post} (Δω)— S_{within agents post} (−Δω)/S_{outside agents post}−S_{within agents post} (Δω)/S_{outside agents post}.
7. Resulting in the detection of disease(s) and mapping of the severity of disease(s). The novel treatment and imaging methods for early detection of diseases and disease mapping using CEST MRI of the present disclosure generally work by a sequence of actions or operation (steps). While the novel treatment and imaging methods for early detection of diseases and disease mapping using CEST MRI are described further below, the methods are generally detailed as follows.
Step 1: Acquiring anatomical T₂-image(s) of specific areas of the brain which are areas of focus as expected under the effects of certain particular diseases. For instance, specific areas of the brain include the hippocampus and the cortex in Alzheimer's disease.
Step 2: Acquiring a CEST image (reference image, S_{outside range}) at a specific chemical shift and at a signal frequency outside the range of frequency that decreases magnetization of related proteins.
Step 3 (or contemporaneously with Step 2): Acquiring CEST images at specific chemical shifts and at frequencies that decrease the magnetization of the proteins (S_{within range}).
Step 4: This step continues in case administration of glucose with or without MCTs inhibitor combined with or without N⁺/H⁺ exchange inhibitor, acquiring a CEST image (reference image, S_{outside post}) at a specific chemical shift and at a signal frequency outside the range of frequency that decreases magnetization of related proteins.
Step 5: (or contemporaneously with Step 4) in case administration of glucose with or without MCT inhibitor combined with or without Na⁺/H⁺ exchange inhibitor, acquiring CEST images at specific chemical shifts and at frequencies that decrease the magnetization of the proteins (S_{within post})
Step 6: This step continues in case administration of agents or drugs, acquiring a CEST image (reference image, S_{outside agents post}) at a specific chemical shift and at a single frequency outside the range of frequency that decreases magnetization of related exchangeable protons of the administrated agents.
Step 7: (or contemporaneously with Step 6) in case administration of glucose, acquiring CEST images at specific chemical shifts and at frequencies that decrease the magnetization of the exchangeable protons of the administrated agents (S_{within agents post})
Step 8: To calculate CEST magnetization transfer (MT) ratio, (MTR) asymmetry-agents post, acquiring CEST images at specific chemical shifts and at frequencies that decrease the magnetization of the exchangeable protons (Δω) of the administrated agents and also acquiring CEST images at specific chemical shifts at the opposite side of the same frequency (−Δω) which is decrease the magnetization of the exchangeable protons at frequency (−Δω) of the administrated agents according to MTR_{asym-agent post} (Δω)=S_{within agents post} (−Δω)/S_{outside agents post} S_{within agents post} (Δω)/S_{outside agents post}.

The CEST chemical shift correction is preferred to be done when Bo variation is larger than 8%. Also, all the CEST images (via signals intensities) are normalized to the reference image. For example, acquiring CEST images around 3.5 ppm for amide proton (between 3.1 ppm to 4 ppm with a step size of 0.1 ppm) as S within range and S_{within post images}, and acquiring CEST images around 2.5 ppm for amine proton (between 1 ppm to 3 ppm with a step size of 0.1 ppm) as S_{within range} and S_{within post images}.

The novel treatment and imaging methods for early detection of diseases and disease mapping using CEST MRI of the present disclosure support most of the pulse sequences for CEST images. For example, one may use fast spin-echo (FSE) pulse sequence by using the following parameters: TR=3 s; TE=6.4 ms; FOV=212×190 mm²; matrix size=256×256; slice thickness=4.4 mm; turbo-spin-echo factor=45; and single slice acquisition. The RF saturation section includes a series of four block RF saturation pulses (200 ms duration each and 2 μT amplitude) at 3Tesla for the human, the optimization of the saturation power and duration depend on the imaging target such as using endogenous or exogenous contrast, some agents need higher power and short duration especially that contain amine and hydroxide protons.

To use the novel treatment and imaging methods for early detection of diseases and disease using CEST MRI of the present disclosure, a person working on MRI (an MRI operator) can follow the general steps noted above (Step 1, Step 2, Step 3, Step 4, Step 5, Step 6, Step 7, and Step 8). Thus, to obtain accurate disease detection and severity mapped results, the MRI operator drives the actions noted in the steps of the methods by control operations with respect to the CEST MRI machine, which captures the images, and by interaction with a computing device, which performs computations, thereby allowing the MRI operator to follow the general steps (Step 1, Step 2, Step 3, Step 4, and Step 5, Step 6, Step 7, and Step 8), the MRI operator will do Step 4 and Step 5 in case there is glucose with or without MCTs inhibitor to get the results. Also, the MRI operator will do Step 6 and Step 7 in case there is administration of agents or drugs to get the results, final, the MRI operator will do Step 8 only to get (MTR) asymmetry-agents post.

The MRI operator may carry out more specific steps. Furthermore, the MRI operator can produce ΔS _{contrast range}, ΔS _{contrast ratio}, ΔSₚₒₛₜ, ΔS_{post ratio}, ΔS_{agents}, ΔS_{agents ratio} MTR_{asym-agent post} and images contrast by using MATLAB (MathWorks) on a computing device and thereby make determinations of diseases that may be present.

For instance, since ΔS _{contrast range}, ΔS _{contrast ratio}, ΔSₚₒₛₜ, ΔS_{post ratio} contrast images, decreased significantly in brain activation and used this method for early detection of the diseases in AD, MS, HD, ALS, cancer and all other neurodegenerative disease by administration of glucose with or without MCTs inhibitors combined with or without Na⁺/H⁺ inhibitors. The ΔS can be used to early detection and identify the severity of the brain activation. The MRI operator can detect many such diseases and map the severity by adhering to the following observations and rules: ΔS contrast can detect and map the distribution of the brain activation in the areas or the structures that are susceptible to early metabolic change such as in Alzheimer's disease before 10-20 years of the symptoms appear, but for controls ΔS contrast will be small change. ΔS contrast can be enhanced by administration of glucose at early detection brain activation, with or without administration of monocarboxylic transporters (MCTs) inhibitor which is combined with or without Na⁺/H⁺ inhibitors, where ΔSₚₒₛₜ and ΔSₚₒₛₜ ratio are images contrast post administration of glucose combined with or without MCTs inhibitor and is decreased significantly with administration of glucose combined with or without MCTs inhibitor and Na⁺/H⁺ inhibitors for early detection of brain activation compared to controls as follow:

operation of the novel treatment and imaging methods for early detection of diseases using CEST MRI after administration of glucose with or without MCT inhibitors combined with or without MCTs inhibitor and Na⁺/H⁺ inhibitors:

1. CEST image (the reference image at S outside post) after administration of glucose with or without MCTs inhibitor and/or Na⁺/H⁺ inhibitors.

2. CEST images (multiple images at S_{within post}) after administration of glucose with or without MCTs inhibitor combined with or without MCTs inhibitor and Na⁺/H⁺ inhibitors.

3. The difference in magnetization (ΔSₚₒₛₜ contrast images after=S_{outside post}−S_{within post}) and or (ΔS_{post ratio} contrast images after=S_{outside post}/S_{within post}) after administration of glucose with or without MCTs inhibitor combined with or without Na⁺/H⁺ inhibitors.

4. Resulting in the detection of disease(s) and mapping of the severity of the disease(s).

ΔS contrast (ΔS _{contrast range}=S_{outside range}−S_{within range}) and/or ΔS contrast ratio (ΔS _{contrast ratio}=S_{outside}/S_{within}) without administration any drug can be used to early detection of brain activation, glycolysis in tissues and senescent tissues, from (ΔS _{contrast range} and/or ΔS _{contrast ratio}) it is possible to map the severity of brain activation, glycolysis in tissues, senescent tissues, and T-cells senescent. (ΔS _{contrast range} and/or ΔS _{contrast ratio}) will be higher in brain activation area, glycolysis in tissues, senescent tissues, and T-cells senescent compared to the normal.

To enhance the contrast in brain activation, glycolysis in tissues, senescent tissues, and T-cells senescent, glucose is administration before 20 mins of administration MCTs inhibitors combined with or without administration of Na⁺/H⁺ exchange inhibitors to enhance the acidification, ΔS_{within final} contrast after is increased significantly for brain activation, glycolysis in tissues, senescent tissues and T-cells senescent, but for the controls (ΔS_{within final} and/or ΔS_{within final ratio}) will be small.

ΔS_{within final} S_{within post}−S_{within}, also; (ΔS_{within final ratio}=S_{within post}/S_{within})

Also, ΔS_{within final} and/or ΔS_{within final ratio} can be used to detect the area under brain activation, glycolysis in tissues, senescent and T-cells senescent early; the ΔS_{within final}, ΔS_{within final ratio} are increased when brain activation, glycolysis in tissues, senescent tissues and T-cells senescent are severe.

Also, in case of injection agents for detection brain activation in neurodegenerative diseases, tissue under glycolysis, senescent tissues, T-cells senescent, hypoxia in tissue, aggressive cancer, blood brain barrier leakage or break down, and the inflammation in cancer

operation of the non-invasive imaging methods for early detection of disease and disease mapping using CEST MRI after administration of agents to detect the diseases by inducing CEST contrast:

1. CEST image (the reference image at S_{outside agents post}) after administration of agents.

2. CEST images (multiple images at S_{within agents post}) after administration of agents.

3. The difference in magnetization (ΔS_{agents} contrast images after=S_{outside agents post}−S_{within agents post}) and/or (ΔS_{agents ratio} contrast images after=S_{outside agents post}/S_{within agents post}) after administration of agents.

4. To calculate CEST magnetization transfer (MT) ratio, (MTR) asymmetry-agents post, acquiring CEST images at specific chemical shifts and at frequencies that decrease the magnetization of the exchangeable protons (Act)) of the administrated agents and also acquiring CEST images at specific chemical shifts at the opposite side of the same frequency (−Δω) which is decrease the magnetization of the exchangeable protons of the administrated agents AT frequency (−Δω) according to MTR_{asym-agent post} (Δω)=S(Δω)t (−Δω)/S_{outside agents post}−S_{within agents post} (Δω)/S_{outside agents post}.

5. Resulting in the detection of disease(s) and mapping of the severity of the disease(s).

ΔS_{agents}=S_{outside agents post}−S_{within agents post}, also; (ΔS_{agents ratio} contrast images=S_{outside agents post}/S_{within agents post})

Also, ΔS_{agents}, ΔS _{agents ratio} and MTR_{asym-agent post} can be used to detect the brain activation in neurodegenerative diseases, tissue under glycolysis, senescent tissues, T-cells senescent, hypoxia in tissue, proliferation, aggressive cancer, blood brain barrier leakage or break down, and the inflammation such as the inflammation in cancer for each one of the diseases the ΔS_{agents} and ΔS _{agents ratio} are increased compared to the controls. Agents administration for imaging the brain activation in neurodegenerative diseases, tissue under glycolysis, aggressive cancer, and senescent tissues, T-cells senescent, these agents like NSAIDs such as sodium salicylate, aspirin; polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate; flavonoids such as luteolin, quercetin, rutin, taxifolin, resveratrol, myricetin, rhein, and others; congo red and it's analogs such as chrysamine-g; nordihydroguaiaretic acid; tannins from brown algae such as phlorotannins include eckol and it's derivatives and most polyphenols of seaweed, and others, these drugs can be accumulated in the inflammation tissue and induce CEST contrast to detect the inflammation and high expressed MCTs cells or tissues in cancer.

Also, ΔS _{agents}, ΔS _{agents ratio} and MTR_{asym-agent post} can be used to detect the hypoxia in tissue, blood brain barrier leakage or break down for each one of above disease the ΔS _{agents}, ΔS _{agents ratio} and MTR_{asym-agent post} are increased after administration of agents compared to the controls, as the disease is severe, the ΔS _{agents}, ΔS _{agents ratio} and MTR_{asym-agent post} are increased for each of proliferation, hypoxia in tissue, aggressive cancer, and senescent tissues, T-cells senescent, blood brain barrier leakage or break down.

Imaging of hypoxia in tissue by CEST MRI by administration of agents such as compounds having the nitro group, —NO2, attached to carbon, also, these agents have one or more of exchangeable protons that can be saturated to induce CEST contrast such as amide, amine, hydroxide protons, and other protons. These agent like Metronidazole, Tolcapone, Nitroxoline, Chloramphenicol, Oxamniquine, Secnidazole and other drugs.

Imaging of blood brain barrier (BBB) leakage or break down by CEST MRI by administration of agents that cannot cross normal BBB or agent cross normal BBB very slowly, these compounds induce CEST contrast can be detected by MRI to imaging and detection BBB leakage or break down. Also, this detection can be used for early detection of disease which are BBB leakage is hallmark of these diseases such as meningitis, brain abscess, epilepsy, multiple sclerosis, neuromyelitis optica (NMO), de vivo disease, Alzheimer's disease, HIV encephalitis, systemic inflammation, brain cancer. Compounds cannot cross normal BBB are: cariporide, morphine, ascorbic acid (vitamin C), carbidopa, dopamine, betaine, congo red and other drugs, which can be used to detect BBB leakage or breakdown at time limit of imaging, such as after 15 mins, 30 mins, 60 mins, or more of administration of the drugs also, compounds that slowly cross BBB or cannot cross BBB readily, can be used to detect BBB leakage or breakdown at time limit of imaging, such as after 30 mins, 60 mins, or more of administration of the drugs such nipecotic acid, guvacine, and other drugs. These agents have one or more of exchangeable protons that can be saturated to induce CEST contrast such as amide, amine, hydroxide protons, and other protons

Imaging of proliferation in tissue by CEST MRI by administration of agents such as agents have CEST contrast by saturation of their exchangeable protons such as thymidine and its analogs.

Neuroinflammation results in a significant decrease intracellular and extracellular pH, methods of treatment nervous system diseases and neurodegenerative diseases, such as, traumatic brain injury, ischemic stroke, epilepsy, Parkinson's disease, ALS, MS, Alzheimer's disease (AD) and other diseases. The common characteristics of these diseases are decreased pH or acidosis, aging or ischemia may cause intracellular and extracellular acidification. Methods of treatment nervous system diseases and neurodegenerative diseases by increase of intracellular pH (pHᵢ) and extracellular pH (pHₑ), methods of increase pHᵢ can treat neurodegenerative and nervous system diseases include by administration of drugs combined with or without glucose, some drugs target and inhibit two or more mechanisms in the same time, the method of treatment comprising administration of pyruvate dehydrogenase kinase (PDK) inhibitors such as sodium phenylbutyrate (4-PBA), sodium dichloroacetate, resveratrol, dicumarol, and other analogs and at least one of:
(i). Reactive oxygen species (ROS) scavengers, such as vitamins A, C, E, resveratrol, and others.
(ii). Administration of carbonic Anhydrases (CA) activators, such as phenylalanine, citalopram, histamine, sertraline, fluoxetine and other drugs.
(iii). Administration of agents or drugs enhance glucose uptake, such as flavonoids such as luteolin, quercetin, and others; polyphenols such as curcumin, rosmarinic acid, epigallocatechin gallate, and others; metformin, phenfoiuiin and other drugs; Sodium Phenylbutyrate (4-PBA), sodium dichloroacetate, resveratrol, dicumarol, and other analogs.
(iv). LDH-A (lactate dehydrogenase A) inhibitors like galloflavin and other drugs.
(v). Increase pHₑ by administration agents contain bicarbonate such as sodium bicarbonate (NaHCO₃) and other agents.

wherein those of (i) through (v) that are in the formulation drugs are in amounts effective in combination administration of pyruvate dehydrogenase kinase (PDK) inhibitors to induce selective increase pHᵢ and pHₑ in nervous system diseases and neurodegenerative diseases relative to untreated same diseases.

Cellular senescence is an aging mechanism that has been included in many age-related diseases and is cause of tissue dysfunction. senescence-associated secretory phenotype (SASP)-mediated tissue damage. Therapeutic targeting of an aging mechanism such as cellular senescence may have an impact on disease and more effective in preventing the progression of aging diseases. also, there is currently no effective way to remove or kill the senescent cells and/or T-cells senescent. Methods of treatment cellular senescence and senescent cells and/or T-cells senescent in many age-related diseases include administration of drugs combined with or without administration of glucose, such as:
1. GLUT (glucose transporter) inhibitors such as phloretin, fasentin, quercetin, EGCG and other drugs.
2. Na⁺/H⁺ exchange inhibitors, such as cariporide, EIPA, amiloride, or another analog active derivative of amiloride.
3. Pyruvate dehydrogenase kinase (PDK) inhibitors, such as Sodium Phenylbutyrate (4-PBA), sodium dichloroacetate, resveratrol, quercetin dihydrate, dicumarol, and other analogs.
4. Carbonic Anhydrases (CA) inhibitors, acetazolamide, methazolamide, and other analogs
5. EGFR (epidermal growth factor receptor) proteins inhibitors like erlotinib, gefitinib, afatinib, cetuximab, necitumumab, and other analogs.
6. Monocarboxylate transporter (MCTs) inhibitor, flavonoids such as luteolin, quercetin, and others; polyphenols such as curcumin, rosmarinic acid, epigallocatechin gallate, and others; cinnamate, a-cyano-4-hydroxycinnamate (4-CIN), lonidamine, diclofenac, syrosingopine, atorvastatin, simvastatin, and other analogs.
7. HK (hexokinase) inhibitors like 3-bromopyruvate, 2-deoxyglucose, lonidamine, chrysin, resveratrol and other analogs.
8. PKM2 (pyruvate kinase M2) inhibitors such as resveratrol, apigenin and other drugs.
9. LDH-A (lactate dehydrogenase A) inhibitors like galloflavin and other drugs.
10. GLS (glutaminase) inhibitors such as CB-839, and BPTES (bis-2-(5-phenylacetamido-1,2,4-thiadiazol-2-yl) ethyl sulfide 3) and other drugs
11. Anti inflammation drugs (NSAIDs) drugs like sodium salicylate, aspirin, diclofenac, celecoxib and other drugs.
12. Mitochondria complex I inhibitors such as rotenone, metformin, phenformin and other drugs.
13. Mitochondria complex II inhibitors, such as atpenins, HQNO, carboxin and TTFA and other analogs.
14. Mitochondria complex III inhibitors, such as Antimycins, Myxothiazol, stigmatellin, strobilurins, Qo inhibitors, strobilurin, propylhexedrine, atovaquone and other drugs.
15. Mitochondria complex IV inhibitors, such as sodium azide, cyanide, and other drugs.
16. Mitochondria complex V inhibitors (ATP synthesis inhibitors) such as pantoprazole, omeprazole, dexlansoprazole, esomeprazole, a bafilomycin, dorafem, rabeprazole, and it's analog and other drugs.
17. The proton pump inhibitors, comprises a vacuolar ATPase inhibitor (V-ATPase), such as pantoprazole, omeprazole, dexlansoprazole, esomeprazole, a bafilomycin, dorafem, rabeprazole, and other analogs.
18. The hyperthermia therapies
19. Chemotherapy administration that induce intracellular acidification, for example cisplatin and it's analog and other chemotherapy that induce intracellular acidification.
20. Mouse double minute 2 homolog (MDM2) inhibitors such as genistein and apigenin and other analogs.

1. Novel Treatment and Imaging Methods for Early Detection of Diseases comprising:
acquiring, by way of a magnetic resonance imaging (MRI) machine, a T₂-image as an anatomical image;
acquiring, by way of the MRI machine, a CEST reference image at a particular Soutside range chemical shift and at a signal frequency outside a range of frequency that decreases magnetization of related proteins;
acquiring, by way of the MRI machine, a plurality of CEST images at a plurality of specific Swithin range, Swithin post, Soutside range, Soutside post, and chemical shifts and frequencies that decrease the magnetization of the related proteins;
calculating a difference in magnetization as (ΔS contrast range) and (ΔS post) between Soutside range and Swithin range and between Soutside post and Swithin post respectively, also, (ΔS contrast ratio) (ΔS post ratio) between Soutside range and Swithin range and between Soutside post and Swithin post at each specific chemical shift and frequency for each image in the plurality of CEST images; calculating the difference and the ratio in magnetization as (ΔS within final) and (ΔS within final ratio) respectively between S within post and Swithin range at each specific chemical shift and frequency for each image in the plurality of CEST images;
and detecting disease and mapping disease severity based on the calculated contrast differences in magnetization before and after administration of glucose with or without monocarboxylic transporters (MCTs) inhibitor combined with or without administration of Na⁺/H⁺ inhibitors.

2. Novel Treatment and Imaging Methods for Early Detection of Diseases comprising:
acquiring, by way of a magnetic resonance imaging (MRI) machine, a T₂-image as an anatomical image;
acquiring, by way of the MRI machine, a CEST reference image at a particular Soutside agents post chemical shift and at a signal frequency outside a range of frequency that decreases magnetization of the agent's protons;
acquiring, by way of the MRI machine, a plurality of CEST images at a plurality of specific Swithin agents post and chemical shifts and frequencies that decrease the magnetization of the agent's protons;
calculating the difference and/or ratio in magnetization as (ΔS agents) and (ΔSagents ratio) between Soutside agents post and Swithin agents post at each specific chemical shift and frequency for each image in the plurality of CEST images; and
detecting disease and mapping disease severity based on calculating the difference and/or ratio in magnetization as (ΔSagents) and (ΔSagents ratio) after administration of agents.

3. Novel treatment and imaging methods of claim 1, wherein detecting disease and mapping disease severity comprises early detection of the brain activation, glycolysis in tissue, senescent cells, inflammation in tissue, immune cells senescent such as T cells senescent.

4. Novel treatment and imaging methods of claim 2, wherein detecting disease and mapping disease severity comprises early detection of the brain activation, glycolysis in tissue, hypoxia, proliferation, senescent cells, inflammation in tissue, immune cells senescent such as T cells senescent, blood brain barrier leakage, and aggressive cancer.

5. Novel treatment and imaging methods of claim 2, comprises, detecting disease and mapping disease severity comprises early detection of brain activation, glycolysis, hypoxia, senescent cells, immune cells senescent such as T cells senescent, proliferation in cancer, blood brain barrier leakage, inflammation in cancer, and aggressive cancer using MTR_{asym-agent post} (Δω) by administration of agents induce contrast in these diseases can be detected by MRI.

6. Novel treatment and imaging methods comprises, methods of treatment cellular senescence, senescent cells, and immune senescence and T cells senescence in many age-related diseases by remove senescent cells and immune senescence and T cells senescence from live tissue by administration of drugs with or without glucose, sometimes one drug can target and inhibit two or more mechanisms in the senescent cells, the method of treatment comprising at least two of list from (a) through (k):
(a). EGFR (epidermal growth factor receptor) proteins inhibitors.
(b). Na+/H+ exchange inhibitors and/or Pyruvate dehydrogenase kinase (PDK) inhibitors.
(c). Chloride-bicarbonate (Cl/HCO3) exchange inhibitors and/or Carbonic Anhydrases (CA) inhibitors.
(d). GLUTs (glucose transporters) inhibitors and/or Monocarboxylate transporter (MCTs) inhibitors.
(e). HK (hexokinase) inhibitors and/or PKM2 (pyruvate kinase M2) inhibitors.
(f). LDH-A (lactate dehydrogenase A) inhibitors and/or GLS (glutaminase) inhibitors.
(g). Anti inflammation drugs (NSAIDs) and/or MD2M inhibitors.
(h). The hyperthermia therapies and/or Chemotherapy administration that induce intracellular acidification.
(i). Mitochondria complex I inhibitors and/or mitochondria complex II inhibitors.
(j). Mitochondria complex III inhibitors and/or mitochondria complex IV inhibitors.
(k). The proton pump inhibitor, comprises a vacuolar ATPase inhibitor (V-ATPase) and/or mitochondria complex V ((ATP synthesis) inhibitors.
Wherein those of (a) through (k) that are in the formulation drugs are in amounts effective in combination to induce selective acidification in cellular senescence and senescent cells relative to nonsenescent cells (normal cells).

7. The formulation of claim 6, comprising at least three of (a) through (k).

8. The formulation of claim 6, comprising at least four of (a) through (k).

9. The formulation of claim 6, comprising at least five of (a) through (k).

10. The formulation of claim 6, comprising at least six of (a) through (k).

11. The formulation of claim 6, comprising at least seven of (a) through (k).

12. The formulation of claim 6, comprising at least eight of (a) through (k).

13. The formulation of claim 6, comprising at least nine of (a) through (k).

14. The formulation of claim 6, comprising at least ten of (a) through (k).

15. The formulation of claim 6, comprising at least eleven of (a) through (k).

16. Novel treatment and imaging methods comprises, methods of treatment nervous system diseases and neurodegenerative diseases, such as, traumatic brain injury, ischemic stroke, epilepsy, Parkinson's disease, ALS, MS, Alzheimer's disease (AD) and other diseases by increase of intracellular pH, which comprising administration of pyruvate dehydrogenase kinase (PDK) inhibitors such as sodium phenylbutyrate (4-PBA), sodium dichloroacetate, and other analogs, combined with or without glucose administration and at least one of:
(i). Reactive oxygen species (ROS) scavengers
(ii). Administration of agents or drugs enhance glucose uptake by cells
(iii). LDH-A (lactate dehydrogenase A) inhibitors like galloflavin and other drugs.
(iv). Administration of carbonic anhydrases (CA) activator
(v). Increase extracellular pH by administration agents contain bicarbonate such as sodium bicarbonate (NaHCO3) or other drugs that increase extracellular pH.
wherein those above of (i) through (v) that are in the formulation drugs are in amounts effective in combination administration of pyruvate dehydrogenase kinase (PDK) inhibitors to induce selective increase intracellular pH and extracellular pH in nervous system diseases and neurodegenerative diseases relative to untreated same diseases, sometimes one drug can target and inhibit two or more mechanisms in nervous system diseases and neurodegenerative diseases.

17. The formulation of claim 16, comprising at least two of (i) through (k).

18. The formulation of claim 16, comprising at least three of (i) through (k).

19. The formulation of claim 16, comprising at least four of (i) through (k).

20. The formulation of claim 16, comprising at least five of (i) through (k).
